(19) United States
(12) Patent Application Publication
(10) Pub. No.: US 2021/0308429 A1
(43) Pub. Date: Oct. 7, 2021

(54) Digestive tract hemorrhage control tube (Li - Van Thiel Tube)

(71) Applicant: Shilun David Li, Excelsior, MN (US)

(72) Inventor: Shilun David Li, Excelsior, MN (US)

(21) Appl. No.: 16/836,965

(22) Filed: Apr. 1, 2020

(51) Int. Cl. A61M 25/09; A61M 25/01

(52) U.S. Cl. CPC A61M 25/09; A61M 25/0158; A61M 2205/073; A61M 2025/0177; A61M 2025/09166; A61M 2025/0915; A61M 2025/09008

(57) ABSTRACT

This medical device (Li-VanThiel tube) is designed to add 21st century technologies to a 20th century device in order to ease application and increase safety. The current Shengstaken-Blakemore tube is very difficult to use and dangerous to deploy. The Li-VanThiel tube utilizes guided tube insertion, inflates by using a pressure regulated pre-charged air canister, It has a smaller pre-deployment diameter and a variable stiffness tube feature to overcome the short comings of the old design.

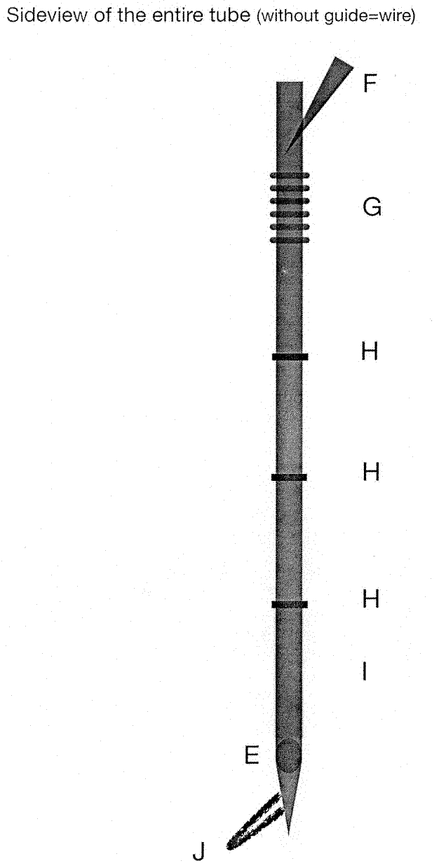

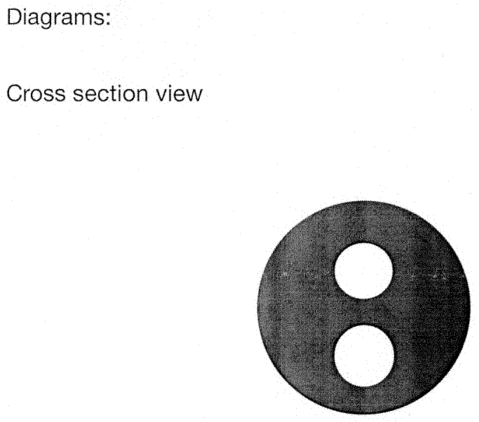

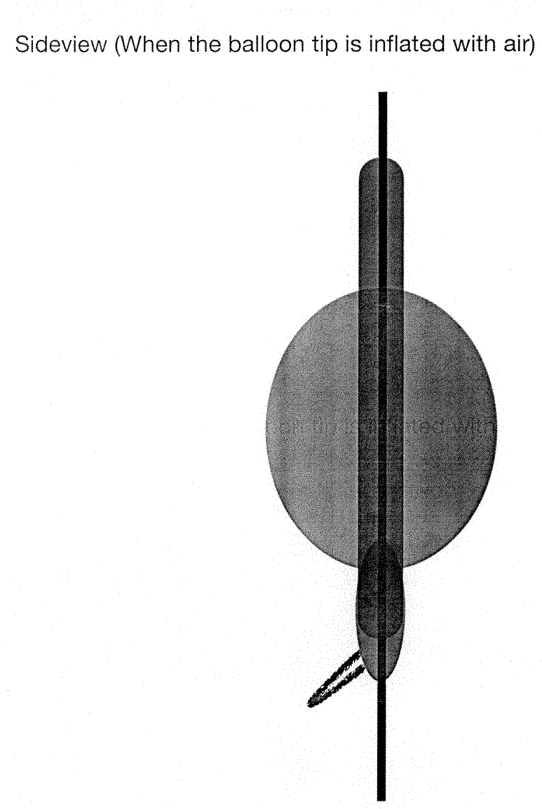

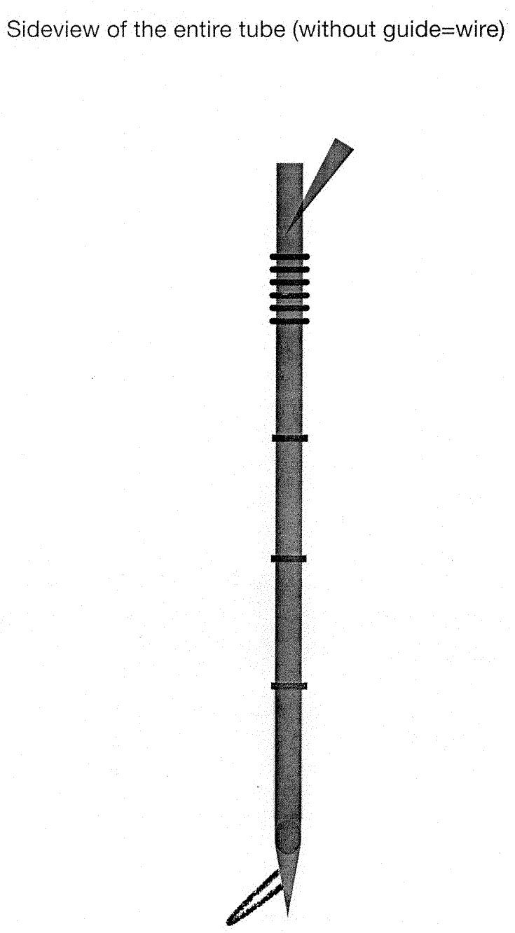

### REASONS FOR THE IMPROVEMENT

A. Ease of Tube Insertion.
1. The Shengstaken-Blakemore tube (SBT) has too many ports.
2. The ports require manual insufflation and pressure monitoring.
3. The ports have to be manually shut using clamps.
4. The current SBT is too large to insert easily.
5. The current SBT is too soft and flimsy which can become a hazard during insertion due to coiling in the month above the upper esophageal sphincter or coiling in the esophagus just above the lower esophageal sphincter.
6. The current SBT has no slippery coating to ease the insertion.
7. The current SBT is blindly inserted during medical emergency.
8. The current SBT has caused unnecessary and potential life threatening trauma by either rupturing the hypo-pharynx or esophagus when the balloon is inserted or inflated.
9. The current SBT has caused unnecessary death as a result of incorrect placement and inappropriate inflation.
10. The current SBT is very difficult to use, as a result, the tube is only used by experienced Hepatologists or trained Gastroenterologists who are familiar with the SBT and its flaws.

### SAFETY ISSUES

1. The Shengstaken-Blakemore tube is inserted blindly and was developed prior to the wide-use of endoscopy.
2. The current tube has caused esophageal trauma, rupture of the hypo-pharynx and esophagus.
3. The current tube has caused death not directly related the disease for which it is used, but as a result of improper placement and inflation.

### New Technologies for the Twenty-First Century

1. Implementing guidance technology: guide-wire insertion, magnetometer guided insertion, direct endoscope guidance and fluoroscopy guidance.
2. Reduction of balloon ports.
3. Automatic pressure regulated balloon inflation.
4. Built-in anchoring features to prevent migration.
5. Composite materials to provide variable stiffness.
6. Simple to deploy.
7. Slippery coating to improve ease of insertion.
8. Can be inserted by all medically trained personnel rather than being limited to highly trained specialists.

### SPECIFICATIONS

1. Tube diameter: 10 mm.
2. Length: 60 cm
3. One balloon port: Balloon is flat around the tube prior to inflation.
4. Guide-wire port.
5. Radio-opaque markings.
6. Visible length markings on tube surface.
7. Pre-charged balloon canister with pressure gauge.
8. Embedded neodymium magnet at the tip.
9. Silicone coating.
10. Variable stiffness with soft curved tip and stiff mid-body design to prevent curling.
11. Loop wire at the tip for endoscopic guided insertion.
12. Multiple notches at the proximal end to anchor the tube at the specially designed bite block with a pre-determined tension spring.
13. Prepackaged with battery powered magnetometer and visual distance reading to detect position of the magnet.

### Applications

Emergency control of massive upper digestive tract bleeding caused by either cirrhosis complicated by proximal gastro/distal esophageal varices or by Mallory-Weiss tears.
Any hospital or rural emergency room to stabilize the patient prior to transfer to a tertiary care center.
Easy instructions for deployment by any of the following professionals: emergency medical staff, ICU staff, surgeons, hepatologists, and gastroenterologists.
The application requires prior airway endotracheal intubation for airway security.
Ease and simplicity of tube utilization as the tube is designed to avoid staff confusion during medical emergency.
Wide range of guidance technologies are included, such as built-in magnet with portable magnetometer, guide-wire port, radio-opaque coating, wire-loop for endoscope attachment to provide direct guidance for proper and safe insertion.
Smaller diameter and stiffer mid-body with materials to prevent trauma as a result of curling inside the esophagus, and perforation or rupture of the esophagus.
Low cost and effective way to stabilize the patient prior to transfer to a tertiary care center where specialists are available.

Clinical Scenarios: The following are 4 clinical scenarios for using this invention to stop upper digestive tract bleeding. All scenarios require first intubation of the patient to obtain airway control to prevent blood aspiration.

1. Insertion Using the Endoscope Guidance:
A. Once the endoscope has reached inside the gastric cavity by direct visualization, a 0.035 guide wire is passed through the working channel. The endoscope is then exchanged over the guide wire. With the guide wire coiled inside the gastric cavity, the Li-VanThiel tube is guided into the stomach. The guide wire is then removed through the wire channel of the tube and the balloon is inflated to pre-determined diameter using a pre-charged air canister. The tube is pulled back to exert pressure against varices at the gastro-esophageal (GE) junction.
B. Using biopsy forceps through the working channel, the loop wire is grasped at the tip of the endoscope. The endoscope is then advanced into the stomach under direct visualization. The balloon is then inflated using pre-charged air canister. The biopsy forceps is released. The scope and the forceps are removed in one piece. The catheter is pulled back to exert pressure against varices at the GE-junction.

2. Insertion using fluoroscopy (real-time x-ray) guidance: The tube is lubricated and inserted through the oral cavity and guided by fluoroscopy during tube advancement. Once the tube tip reached gastric cavity, the balloon is inflated using pre-charged air canister and pulled back against the GE-junction.

3. Insertion using portable magnetometer guidance. The tube is lubricated and inserted through the oral cavity and guided by a pocket-sized magnetometer within the kit. The magnetometer can follow the tip into the epigastric region. The balloon is then inflated and tube pulled back against the GE-junction.

### DEVICE DESCRIPTION

This device has 2 ports. One port is for the guide-wire to ease insertion into the stomach. The second port is to inflate the distal balloon.

The device is designed to be inserted into the patient through the mouth. The tube can be guided using an endoscope by a guide-wire/loop string or by using realtime x-ray imaging to visualize the radio-opaque markings every 10 cm. Alternatively, it can be inserted like an oral-gastric tube and guided by a hand-held magnetometer because of an embedded N50 neodymium magnet at the tip.

Once the distal tube has reached beyond the lower esophageal sphincter, the balloon will be inflated to 10 cm and pulled back against the gastro-esophageal junction to tamponade the bleeding varix. The tube then is secured onto a proprietary bite block with grooves that can lock this device while exerting pressure against the GE-junction by the inflated balloon.

1. The Li-VanThiel tube is safer to use than the SBT.

2. The Li-VanThiel tube is easier to insert than the SBT.

3. The Li-VanThiel tube is more versatile than the outdated SBT design.
